(19) United States
(12) Patent Application Publication
(10) Pub. No.: US 2026/0256581 A1
(43) Pub. Date: Sep. 3, 2026

(54) A DELIVERY CATHETER SYSTEM

(71) Applicant: Medtronic, Inc., Minneapolis, MN (US)

(72) Inventor: Erik C. Griswold, Penngrove, CA (US)

(73) Assignee: Medtronic, Inc., Minneapolis, MN (US)

(21) Appl. No.: 18/875,504

(22) PCT Filed: Jun. 12, 2023
(86) PCT No.: PCT/IB2023/056056

§ 371 Date: Dec. 16, 2024

Related U.S. Application Data
(60) Provisional application No. 63/353,067, filed on Jun. 17, 2022.

(51) Int. Cl. A61F 2/24

(52) U.S. Cl. CPC A61F 2/2436

(57) ABSTRACT

A delivery catheter system is provided with a catheter assembly including an elongate catheter body having a distal tip portion and a capsule distal to the distal tip portion, said capsule comprising a capsule body capable of containing all or part of a deliverable prosthesis. The system further includes a locating member comprising a first portion connected to the capsule body, and an annular second portion extending from the first portion and configured for contacting a valve annulus. The annular second portion is moveable from a collapsed state, in which the second annular portion is at least partially retained within a sheath, to a deployed state in which the second annular portion extends radially outwardly from the capsule body.

### FIELD

The present teachings relate to a delivery catheter system.

### BACKGROUND

Prosthetic heart valves that are collapsible to a relatively small circumferential size can be delivered into a patient via a catheter. Catheters have long been used for the treatment of diseases of the cardiovascular system, such as treatment or removal of stenosis. More recently, catheters have been used for replacement of heart valves, for example: the aortic valve in a procedure sometimes known as transcatheter aortic valve implantation (TAVI); the mitral valve in a procedure sometimes known as transcatheter mitral valve replacement (TMVR); or the tricuspid valve in a procedure sometimes known as transcatheter tricuspid valve replacement (TTVR).

Collapsible prosthetic heart valves typically take the form of a valve structure mounted on a stent. When a collapsed prosthetic valve is positioned at or near the annulus of the patient's heart valve that is to be replaced, the prosthetic valve can be deployed from the delivery catheter system by allowing the prosthetic valve to expand as a sheath covering the valve is withdrawn.

Prior to deploying the prosthetic valve, the catheter is required to extend through the valve annulus. In some procedures, however, for example during transcatheter tricuspid valve replacement, the available space behind the valve annulus may be limited and/or may have a complex shape.

There is a need to provide an improved delivery catheter assembly for the delivery of prosthetic valves. The present teachings seek to overcome or at least mitigate one or more problems associated with the prior art.

### SUMMARY

According to a first aspect, there is provided a delivery catheter system comprising: a catheter assembly including an elongate catheter body having a distal tip portion and a capsule distal to the distal tip portion, said capsule comprising a capsule body capable of containing all or part of a deliverable prosthesis; a locating member comprising a first portion connected to the capsule body, and an annular second portion extending from the first portion and configured for contacting a valve annulus; and wherein the annular second portion is moveable from a collapsed state, in which the second annular portion is at least partially retained within a sheath, to a deployed state in which the second annular portion extends radially outwardly from the capsule body.

It will be appreciated that the delivery catheter assembly may be used for a range of different purposes, and the article being delivered will vary accordingly. The delivery catheter assembly may be used for a transcatheter tricuspid valve implantation, or a transcatheter mitral valve implantation, a transcatheter aortic valve implantation, or transcatheter pulmonic valve implantation and, as such, the article may be a replacement valve. Alternatively, the delivery catheter system may be used for any type of transcatheter valve replacement or repair procedure that requires locating a valve annulus.

Arranging the locating member such that the second annular portion extends radially outwardly from the capsule body in the deployed state has been found to facilitate positioning of the capsule body at the valve annulus.

The first portion of the locating member may be connected to an external surface of the capsule body.

Connection of the locating member to the outside of a capsule enables the position of the locating member to be adjusted to suit the procedure being performed. This can be particularly advantageous as it enables the distance by which the capsule needs to extend through a valve to be repaired to be adjusted, for example in circumstances where space is limited behind a valve to be replaced (such as in the right ventricle behind the tricuspid valve).

For example, the locating member may be positioned on the capsule body such that a distal edge of the second annular portion is at least a quarter of the way from the proximal end towards a distal end of the capsule body, for example at least half-way from the proximal end towards the distal end.

The first portion of the locating member may be connected to an external surface of the capsule body such that a distal edge of the annular second portion is spaced apart from a proximal end of the capsule body by a first distance.

Positioning the flexible locating member away from the proximal end of the capsule may reduce the distance by which the capsule needs to extend through a valve to be repaired to correctly position the prosthesis.

The distal edge of the annular second portion may be closer to a distal end of the capsule body than the proximal end when the second portion is in the deployed state.

Positioning the flexible locating member closer to the distal end of the capsule further reduces the distance by which the capsule needs to extend through a valve to be repaired to correctly position the prosthesis.

The distal edge of the annular second portion in the deployed state may be spaced apart from the distal end of the capsule body by a second distance of less than 20 mm, for example a second distance of less than 15 mm, for example a second distance of less than 10 mm, for example by a second distance of approximately 5 mm.

Positioning of the flexible locating member at these distances away from the distal end of the capsule may be particularly advantageous in facilitating the positioning of a prosthesis.

Relative movement between the sheath and the capsule in a first direction may enable the annular second portion of the locating member to move from the collapsed state to the deployed state.

It will be appreciated that the sheath may be formed by the catheter body, or may be provided as a separate sheath positioned within the catheter body and fully or partially extendable from the distal tip portion of the catheter assembly.

A proximal retraction of the sheath relative to the capsule and/or a distal movement of the capsule relative to the sheath may enable the locating member to move from the collapsed state to the deployed state.

Deployment of the locating member via a proximal retraction of the sheath and/or a distal movement of the capsule relative to the sheath reduces the amount of extension of the capsule required. This can be particularly advantageous as it enables the distance by which the capsule needs to extend through a valve to be repaired to be adjusted, for example in circumstances where space is limited behind a valve to be replaced (such as in the right ventricle behind the tricuspid valve).

The delivery catheter system may comprise a deployment arrangement to deploy the prosthesis from the capsule body, said deployment arrangement comprising an actuating arrangement configured to move the prosthesis in a distal direction and a retraction arrangement configured to proximally retract the capsule body.

Deployment of the prosthesis via a proximal retraction of the capsule body reduces the amount of extension of the capsule required. This can be particularly advantageous as it enables the distance by which the capsule needs to extend through a valve to be repaired to be adjusted, for example in circumstances where space is limited behind a valve to be replaced (such as in the right ventricle behind the tricuspid valve).

Movement of the prosthesis in the distal direction may be restricted to a distance less than an elongate length of the capsule body.

Restriction of the amount of distal extension that the system is capable of reduces the amount of extension that may be required once the capsule body has been positioned at a valve of a patient.

The locating member may be positioned on the capsule body such that a distal edge of the second annular portion is spaced apart from a proximal end of the capsule body by a first distance, and wherein movement of the prosthesis in the distal direction is restricted to a distance substantially equal to the first distance.

The actuating arrangement may comprise a hydraulic actuating arrangement.

The distal edge of the second annular portion may be spaced apart from the distal end of the capsule body by a second distance, and wherein retraction of the capsule body by a distance at least equal to the second distance enables deployment of the prosthesis.

Simultaneous actuation of the of the actuation arrangement and retraction arrangement may be prevented.

The annular second portion may comprise a flexible portion, for example a flexible brim or skirt.

The provision of a flexible locating member enables said locating member to be visible by echolocation, which facilitates positioning of the replacement prosthesis.

The delivery catheter system may comprise an agitation arrangement configured to agitate the flexible portion of the annular second portion.

This arrangement causes movement of the flexible portion of the locating member, which enhances its visibility by echolocation, which facilitates positioning of the replacement prosthesis.

The agitation arrangement may be configured to impart a pulsed movement of the flexible portion of the annular second portion.

This has been found to further enhance visibility of the locating member by echolocation.

The agitation arrangement may comprise one or more tubular members configured to dispense a jet from a distal end of the tubular member towards the flexible portion of the locating member.

The tubular members may dispense a jet of saline solution.

The prosthesis may be moveable from a compressed state within the capsule body to an expanded state upon deployment of the prosthesis from the capsule body.

The first portion of the locating member may be connected to the external surface of the capsule body by adhesive, sewing, crimping or thermobonding.

The second annular portion may be substantially frustoconical.

The prosthesis may comprise one or more anchor members connecting the prosthesis to the capsule, and wherein the anchor members are positioned on a proximal side of the prosthesis.

The locating member may comprise a support frame, e.g. a nitinol support frame, and a porous polymeric membrane covering the support frame.

The support structure may comprise a shape memory material such that the support frame acts as a deployment arrangement to move the locating member from the collapsed state into the deployed state.

According to a second aspect, there is provided an introducer device comprising: an introducer sheath, wherein the catheter body of the first aspect is arranged, e.g. substantially coaxially, within the introducer sheath, in use.

According to a third aspect, there is provided a method of delivering a prosthesis contained within a capsule body of a capsule of a delivery catheter system to a target location within patient's vasculature, the method comprising the steps of: arranging a locating member connected to an external surface of the capsule body in a collapsed state in which the locating member is at least partially retained within a sheath: inserting the delivery catheter system into a patient's vasculature to a desired location; at least partially deploying the locating member by a proximal retraction of the sheath; aligning the locating member with a valve annulus; and deploying the prosthesis into position within the valve annulus using a deployment arrangement.

The method may comprise the step of moving the prosthesis in a distal direction using an actuating arrangement to deploy the prosthesis from the capsule body.

The method may comprise the step of proximally retracting the capsule body via a retraction arrangement to deploy the prosthesis from said capsule body.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Embodiments will now be described with reference to the accompanying drawings, in which:

FIG. 1 is a schematic cross-sectional side view of a delivery catheter system according to an embodiment, with a locating member in a collapsed state;

FIG. 2 is a schematic cross-sectional side view of the delivery catheter system of FIG. 1, with the locating member in a deployed state;

FIG. 3 is a side view of a locating member and a capsule of the delivery catheter system of FIG. 1;

FIG. 4 is a schematic cross-sectional side view of the delivery catheter system of FIG. 1;

FIGS. 5, 6 and 7 show implantation of a prosthetic valve using the delivery catheter system;

FIGS. 8 and 9 show implantation of a prosthetic valve using the delivery catheter system; and

FIG. 10 is a side view of the delivery catheter system.

### DETAILED DESCRIPTION

The human heart includes right and left atriums, and right and left ventricles. Disposed between the left atrium and the left ventricle is the mitral valve. As left atrial pressure increases above that of left ventricle, the mitral valve opens and blood passes into the left ventricle. The mitral valve is formed from a pair of leaflets having distal edges that meet so as to close the mitral valve. Each leaflet is attached to an annular region of the heart structure known as the valve annulus. Disposed between the right atrium and the right ventricle is the tricuspid valve. As right atrial pressure increases above that of right ventricle, the tricuspid valve opens and blood passes into the right ventricle. The tricuspid valve is formed from three leaflets having distal edges that meet so as to close the tricuspid valve. Each leaflet is attached to an annular region of the heart structure known as the valve annulus. In addition to the mitral valve and the tricuspid valve, the heart includes the aortic valve and the pulmonary valve. The aortic valve permits one-way flow of blood from the left ventricle to the aorta. The pulmonary valve permits one-way flow of blood from the right ventricle to the pulmonary artery. Each of the aortic valve and the pulmonary valve are formed from three leaflets having distal edges that meet so as to close the respective valve. Each leaflet is attached to an annular region of the heart structure known as the valve annulus.

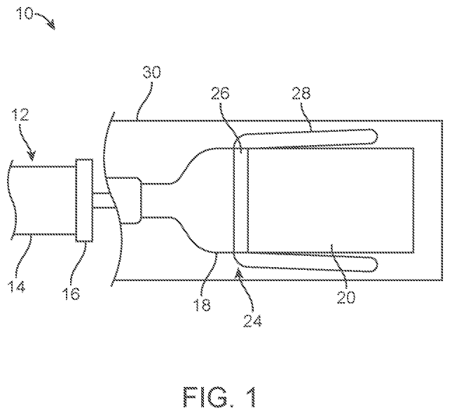

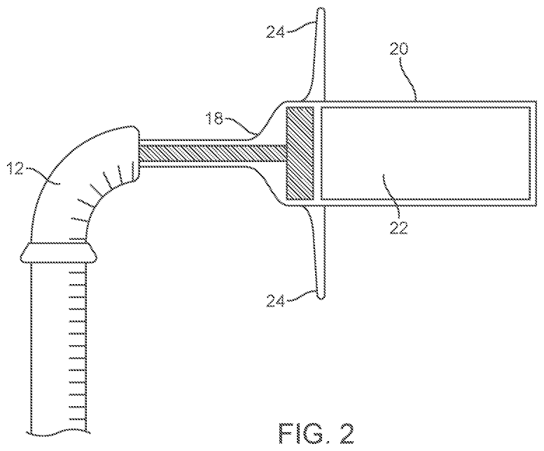

Referring firstly to FIGS. 1 and 2, there is shown a delivery catheter system generally designated 10. The delivery catheter system 10 is intended to deliver an article or prosthesis to a desired location in a patient's vasculature or heart. The delivery catheter system 10 includes a catheter 12 including an elongate catheter body 14 having a distal tip portion 16. The delivery catheter system 10 also includes a capsule 18 shown distal to the distal tip 16, said capsule defining a capsule body 20 capable of containing all or a part of a deliverable prosthesis 22, as shown in FIG. 2. The prosthesis 22 is moveable from a compressed state within the capsule body 20 to an expanded state upon deployment of the prosthesis 22 from the capsule body 20.

In the present arrangement, the delivery catheter system 10 is intended to deliver a prosthetic valve to a valve annulus, for example within a heart of a patient. It will be appreciated that the delivery catheter assembly may be used for a range of different purposes, and the article being delivered will vary accordingly. The delivery catheter assembly may be used for a transcatheter tricuspid valve implantation, or a transcatheter mitral valve implantation and as such the article may be a replacement valve. Alternatively, the delivery catheter assembly may be used for any type of valve replacement procedure.

The delivery catheter system 10 includes a locating member 24 that acts as a guide for the delivery catheter system 10. The locating member 24 guides the operator of the delivery catheter system 10 during implantation such that the capsule 18 device is located at a desired position relative to the native valve annulus. The locating member 24 is connected to a surface of the capsule body 20, such as an external surface of the capsule body. Connection of the locating member 24 to the outside of a capsule enables the position of the locating member 24 to be varied to suit the application, for example in order to adjust the distance by which the capsule is required to extend through a valve to be repaired.

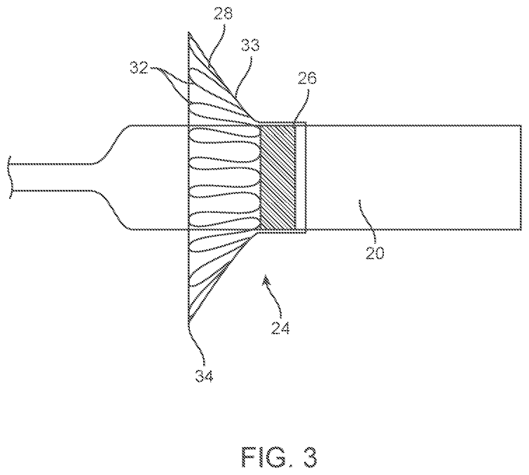

The locating member 24 is connected to the capsule body 20 via a first, or connecting, portion 26. In some embodiments, for example those illustrated in FIG. 3, the locating member 24 is connected to an external surface of the capsule body 20 via a first, or connecting, portion 26. The locating member 24 includes an annular second portion 28 configured for contacting a valve annulus and extending outwardly from the external surface of the capsule body 20. The annular second portion 28 extends radially outwardly from the connecting portion 26. The annular second portion 28 is moveable from a collapsed state, in which the annular second portion 28 is at least partially retained within a sheath 30, to a deployed state, in which the annular second portion 28 is substantially, i.e. at least partially, outside the sheath 30 and extends radially outwardly from the capsule body 20.

The annular second portion 28 is initially provided in an un-deployed or collapsed state whereupon it conforms closely to the outer surface of the capsule body 20, as is illustrated in FIG. 1. The collapsed annular second portion 28 is retained in the collapsed position by the sheath 30. The sheath 30 maintains the annular second portion 28 in the collapsed state during advancement of the delivery catheter system 10 through the vasculature of the patient.

Upon positioning of the capsule 18 in the desired location within a patient, the annular second portion 28 can be moved to the deployed state. This may be achieved by, for example, manipulating the sheath 30 covering the annular second portion 28 in a proximal direction so as to uncover the annular second portion 28. Put another way, this may be achieved by retracting the sheath 30 relative to the capsule 18. Alternatively, or in conjunction, the annular second portion 28 can be moved to the deployed state by advancing capsule 18 distally relative to sheath 30. The annular second portion 28 may then move from the collapsed state to the deployed state, as is illustrated in FIG. 2. Deployment of the locating member 24 via a proximal retraction of sheath 30 is advantageous in certain use cases, because this maneuver reduces the amount of extension of the capsule 18 through the valve annulus that is required as compared to a maneuver in which the capsule 18 is only advanced distally relative to the sheath 30 in order to deploy the locating member 24.

It will be appreciated that the sheath 30 may be provided as an introducer sheath of a separate delivery or introducer device (not shown). In such arrangements, it will be appreciated that the catheter 12 is arranged, e.g. substantially coaxially, within the introducer sheath 30, in use. In alternative arrangements, the sheath 30 may be provided as a separate sheath positioned within the catheter body and extending from the distal tip 16 and over all or part of capsule 18 and locating member 24.

Referring further to FIG. 3, the locating member 24 is illustrated, among other things. The first portion 26 may be provided as a first annular portion. In some alternative arrangements, the first portion 26 may be non-circumferential and so may not extend around an entirety of the circumference of the capsule body 20.

The second annular portion 28 may be substantially frustoconical. For example, the second annular portion 28 of the locating member 24 is provided in the form of a tapered brim or skirt. The second annular portion 28 as depicted in FIG. 3 terminates at a distal end 34 of the second annular portion 28.

The locating member 24 is connected to the external surface of the capsule body 20. The locating member 24 may be connected to the capsule body 20 by any suitable means such as adhesive, sewing, crimping or thermobonding. In the present arrangement, it is the first portion 26 that is connected to the external surface of the capsule body 20.

The annular second portion 28 is flexible, i.e. at least a portion or region of the second portion 28 is flexible. The provision of a flexible locating member enables said locating member 24 to be visible by echolocation, which facilitates positioning of the replacement prosthesis.

The locating member 24 may move from the collapsed state to the deployed state via one or more shape memory portions of the annular second portion 28. The annular second portion 28 may be at least partially formed from a shape memory material, for example a shape memory alloy or shape memory polymer. In the embodiments discussed herein, the shape memory material is nitinol, but it will be appreciated that any suitable shape memory material may be used.

The locating member 24 includes a support frame 32 and a porous polymeric membrane 33 covering the support frame 32. The support frame 32 may be at least partially made from a shape memory material such that the support frame acts as a deployment arrangement to move the locating member 24 from the collapsed state into the deployed state. The support frame 32 may be a nitinol support frame.

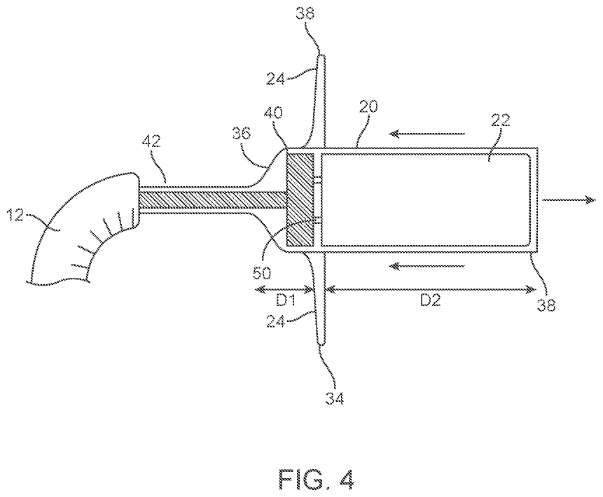

Referring now to FIG. 4, the locating member 24 is positioned on the external surface of the capsule body 20. The locating member 24 is positioned such that the radially outward edge 34 of the second annular portion 28 is spaced apart from a proximal end 36 of the capsule body 20 by a first distance D1. The distal edge 34 of the second annular portion 28 is spaced apart from the distal end 38 of the capsule body 20 by a second distance D2.

In the illustrated arrangement, the locating member 24 is positioned such that a distal edge (i.e. the radially outward edge) 34 of the second annular portion 28 is approximately a quarter of the way from the proximal end 36 towards a distal end 38 of the capsule body 20. Put another way, in the illustrated arrangement, the first distance D1 is approximately a quarter of the length of the capsule body 20 and the second distance D2 is approximately three quarters of the length of the capsule body 20. In alternative arrangements, for example, the locating member 24 may be positioned at any suitable position on the capsule body 20 such that a distal edge 34 of the second annular portion 28 is positioned at any suitable position to suit the application, for example at least a quarter of the distance from the proximal end 36 towards the distal end 38 of the capsule body 20, approximately or at least half-way from the proximal end 36 towards the distal end 38, or at least three quarters of the distance from the proximal end 36 to the distal end 38. Additionally, it will be understood that the distances D1 and D2 may vary depending on the size of the prosthesis 22 and/or capsule body 20.

The delivery catheter system 10 includes a deployment arrangement for deploying the prosthesis 22 from the capsule 18. The deployment arrangement has an actuating arrangement 40 configured to move the prosthesis in a distal direction. The actuating arrangement 40 includes a platform to which the prosthesis 22 is attached. The prosthesis 22 includes one or more anchor members 50 connecting the prosthesis 22 to the actuating arrangement 40. The anchor members 50 are positioned on a proximal side of the prosthesis 22. In the present arrangement, the anchor members 50 attach the prosthesis 22 to the actuating arrangement 40. The actuating arrangement may include a hydraulic actuating arrangement. In some arrangements, the actuating arrangement may be provided in the form of an actuating wire configured to move the prosthesis 22 relative to the capsule body 20.

The deployment arrangement has a retraction arrangement 42 configured to proximally retract the capsule body 20. In some arrangements, it will be appreciated that simultaneous actuation of the of the actuation arrangement and retraction arrangement is prevented. The retraction arrangement may be provided in the form of an actuating wire configured to move the capsule body 20 relative to the prosthesis.

Positioning the radially outward edge 34 of the flexible locating member 24 away from the proximal end 36 of the capsule 20 enables a reduction of the distance by which the capsule 20 is required to extend through a native valve of patient to correctly position the prosthesis 22 prior to partial deployment of said prosthesis 22. For example, the reduction of the distance by which the capsule 20 is required to extend through a native valve of a patient to correctly position the prosthesis 22 may be substantially equal to the first distance D1. This is achieved as it enables the prothesis 22 to be deployed from the capsule body 20 to move from the compressed state within the capsule body 20 to an expanded state via either only a proximal retraction of the capsule body, or via a combination of a proximal retraction and a distal extension. Correspondingly, the second distance D2 is substantially equal to the distance by which the capsule 20 is required to extend through a native valve of a patient to correctly position the prosthesis 22 prior to partial deployment of said prosthesis 22. It will be understood that the first distance D1 and the second distance D2 may be varied to suit the needs of the particular application.

In order to be deployed from the capsule 20, the prosthesis 22 is required to move distally relative to the distal end 38 of the capsule body 20. In order to partially deploy the prosthesis 22 from the capsule body 20, the capsule body 20 is retracted relative to the prosthesis 22. This may be achieved by moving the prosthesis 22 distally via the deployment arrangement by a distance that is substantially equal to the first distance D1. Movement of the prosthesis distally by a distance substantially equal to the first distance D1 correctly positions the partially deployed prosthesis 22 within the leaflets L of the native valve.

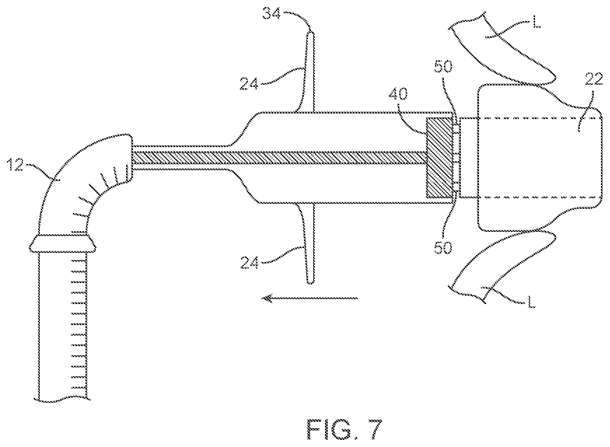

Deployment of the prosthesis via either only a proximal retraction of the capsule body, or via a combination of a proximal retraction and a distal extension may reduce the amount of extension of the capsule 22 through a valve annulus that is required. This can be particularly advantageous as it enables the distance by which the capsule needs to extend through a valve to be repaired to be adjusted, for example in circumstances where space is limited behind a valve to be replaced (such as in the right ventricle behind the tricuspid valve). In order to fully deploy the prosthesis 22 from the capsule body 20, the capsule body 20 is retracted relative to the prosthesis 22 by a distance substantially equal to or at least equal to the second distance D2, as is illustrated in FIG. 7.

In order to limit the amount of extension of the capsule 20 through a valve annulus once the capsule body 20 has been positioned at a native valve of a patient, movement of the prosthesis 22 in the distal direction may be restricted. In the present arrangement, movement of the prosthesis 22 in the distal direction is restricted to a distance less than an elongate length of the capsule body 20. Movement of the prosthesis 22 in the distal direction is restricted to a distance substantially equal to the first distance D1.

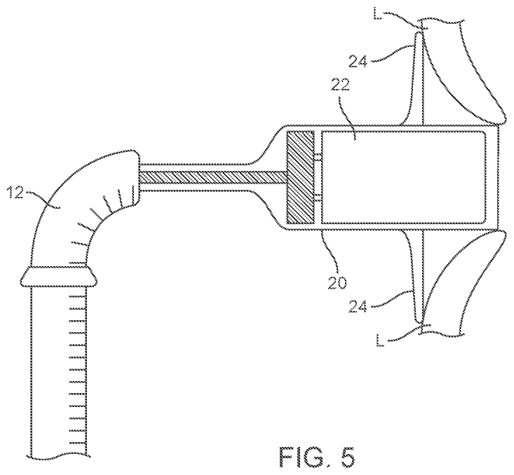

Referring to FIG. 5 to 7, a delivery catheter system 10 is illustrated with the capsule 20 positioned at a native valve annulus within a patient's heart. In particular, the capsule 20 is positioned within a patient's heart such that the annular second portion 28 of the locating member 24 is in contact with the leaflets L of the patient's native valve.

In this arrangement, the locating member 24 is positioned on the capsule body 20 such that a distal edge 34 of the second annular portion 28 is closer to a distal end 38 of the capsule body 20 than the proximal end 36. In some arrangements, it will be appreciated that the distal edge 34 of the second annular portion 28 may be spaced apart from the distal end 38 of the capsule body 20 by a second distance D2 of less than 20 mm, for example a second distance of less than 15 mm, for example a second distance of less than 10 mm, for example by a second distance of approximately 5 mm.

A method of delivering the prosthesis 22 contained within the capsule body 20 of the capsule 18 of a delivery catheter system 10 to a target location within patient's vasculature will now be discussed.

A locating member 24 is connected to the external surface of the capsule body 20 and is initially provided in an un-deployed or collapsed state whereupon it conforms closely to the outer surface of the capsule body 20. The collapsed annular second portion 28 is retained in the collapsed position by the sheath 30.

The delivery catheter system 10 is inserted into a patient's vasculature. The sheath 30 maintains the annular second portion 28 in the collapsed state during advancement of the delivery catheter system 10 through the vasculature of the patient to the desired valve annulus.

Once the delivery catheter system 10 has reached the desired location, the locating member 24 is deployed by a proximal retraction of the sheath 30 relative to the capsule 18. The capsule 18 is then aligned within a valve annulus of a patient using the locating member 24, as is shown in FIG. 5.

Once the capsule has been correctly positioned within the valve annulus, the prosthesis 22 can be deployed within the valve annulus using the deployment arrangement.

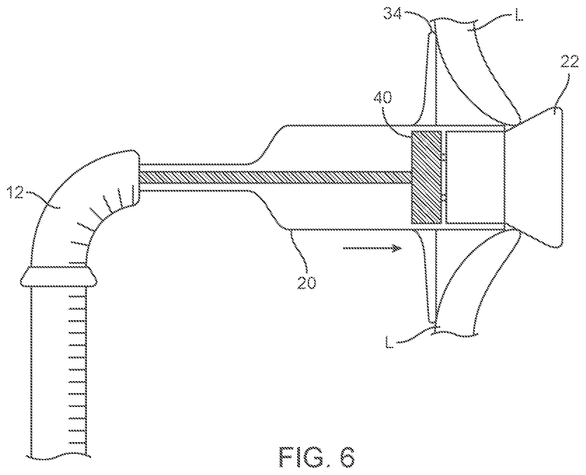

Initially, the prosthesis 22 is moved in a distal direction using an actuating arrangement. This distal movement partially deploys the prosthesis 22 from the capsule body 20, as is shown in FIG. 6. Movement of the prosthesis 22 in the distal direction is substantially equal to the first distance D1. As has been discussed above, movement of the prosthesis 22 in the distal direction may be restricted to a distance that is substantially equal to the first distance D1.

Following the distal extension, the capsule body 20 is proximally retracted in order to fully deploy the prosthesis at the valve annulus, as is illustrated in FIG. 7.

Once deployment of the prosthesis 22 has been completed, the sheath 30 is distally extended relative to the capsule body 20 so as to move the locating member 24 into the collapsed state. The delivery catheter system 10 can then be removed from the patient's vasculature.

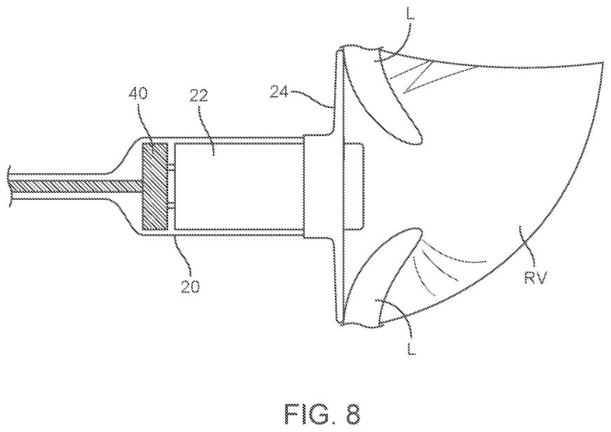

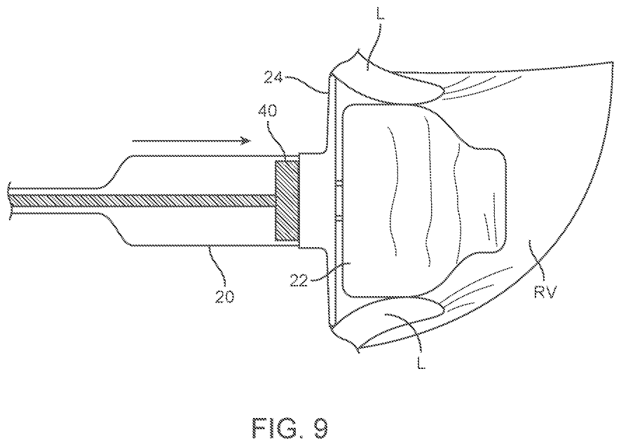

Referring to FIGS. 8 and 9, a delivery catheter system 10 is illustrated with the capsule 20 positioned at a native valve annulus within a patient's heart. In the illustrated arrangement, the capsule 20 is positioned at a native annulus of the tricuspid valve within a patient's heart. The capsule 20 is positioned within a patient's heart such that the annular second portion 28 of the locating member 24 is in contact with the leaflets L of the patient's native valve.

In this arrangement, the locating member 24 is positioned on the capsule body 20 such that a distal edge 34 of the second annular portion 28 is positioned substantially at the distal end of the capsule body 20. In the illustrated arrangement, the positioning of the locating member 24 on the capsule body 20 is such that the capsule 20 does not need to extend through the tricuspid valve and into the right ventricle RV during location of the locating member 24 and or deployment of the prosthesis 22.

A method of delivering the prosthesis 22 contained within the capsule body 20 of the capsule 18 of a delivery catheter system 10 to a target location within patient's vasculature will now be discussed.

A locating member 24 is connected to the external surface of the capsule body 20 and is initially provided in an un-deployed or collapsed state whereupon it conforms closely to the outer surface of the capsule body 20. The collapsed annular second portion 28 is retained in the collapsed position by the sheath 30.

The delivery catheter system 10 is inserted into a patient's vasculature. The sheath 30 maintains the annular second portion 28 in the collapsed state during advancement of the delivery catheter system 10 through the vasculature of the patient to the desired valve annulus.

Once the delivery catheter system 10 has reached the desired location, the locating member 24 is deployed by a proximal retraction of the sheath 30 relative to the capsule 18. The capsule 18 is then aligned within a valve annulus of a patient using the locating member 24, as is shown in FIG. 8.

Once the capsule 18 has been correctly positioned at the valve annulus, the prosthesis 22 can be deployed within the valve annulus using the deployment arrangement.

Due to the positioning of the distal edge 34 of the second annular portion 28 at the distal end 38 of the capsule body 20, deployment of the prosthesis 22 is affected only by the actuating arrangement. Movement of the prosthesis 22 in the distal direction is substantially equal to the first distance D1.

As has been discussed above, movement of the prosthesis 22 in the distal direction may be restricted to a distance that is substantially equal to the first distance D1. In this arrangement, this enables the prosthesis 22 to be fully deployed using only distal extension of the prosthesis 22. As such, no proximal retraction of the capsule body 20 is required for deployment of the prosthesis. It will be appreciated that the deployment of the prosthesis 22 may be carried out in two stages. Put another way, the prosthesis 22 may be moved in a distal direction by the actuating arrangement part way along the length of the capsule body 20 so as to partially deploy the prosthesis 22. Once the prosthesis 22 has been correctly positioned, the prosthesis 22 may again be moved in a distal direction by the actuating arrangement to fully deploy the prosthesis 22.

Once deployment of the prosthesis 22 has been completed, the sheath 30 is distally extended relative to the capsule body 20 so as to move the locating member 24 into the collapsed state. The delivery catheter system 10 can then be removed from the patient's vasculature.

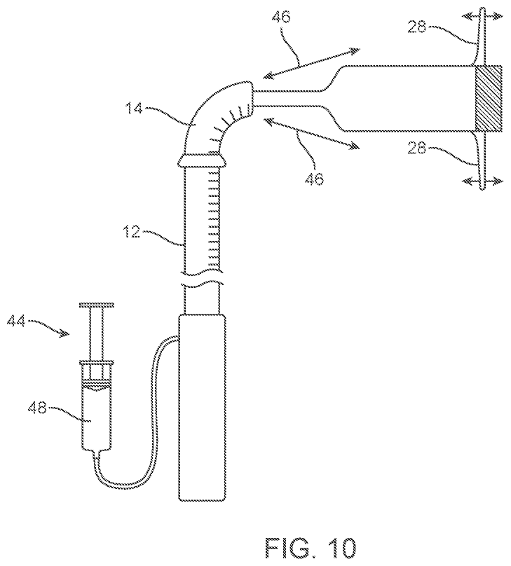

Referring now to FIG. 10, in some arrangements, the delivery catheter system 10 may be provided with an agitation arrangement 44 configured to agitate the flexible portion of the second annular portion 28 of the locating member 24. The provision of an agitating arrangement to agitate the flexible locating member 24 enables said locating member to be visible by echolocation, which facilitates positioning and deployment of the replacement prosthesis 22. The agitation arrangement 44 may be configured to impart a pulsed movement of the flexible portion of the locating member 24 to further enhance visibility of the locating member 24 by echolocation.

In the present arrangement, the agitation arrangement 44 includes one or more tubular members (not shown) configured to dispense a jet 46 from a distal end thereof towards the flexible portion 28 of the locating member 24. The one or more tubular members may dispense a jet of saline solution towards the locating member 24.

The agitation arrangement 44 is also provided with an actuator 48 to dispense the jet 46 from the tubular members. In the present arrangements, the actuator 48 is provided in the form a manually operated syringe. In alterative arrangements, it will be appreciated that the any suitable manual or automatic actuator may be used.

Although the teachings have been described above with reference to one or more preferred embodiments, it will be appreciated that various changes or modifications may be made without departing from the scope as defined in the appended claims.

With regard to the terms “distal” and “proximal” within this description, unless otherwise specified, the terms can reference a relative position of the portions of the delivery catheter system with reference to an operator and/or a location in the vasculature or heart. For example, “proximal” can refer to a position closer to the operator of the device or an incision into the vasculature, and “distal” can refer to a position that is more distant from the operator of the device or further from the incision along the vasculature (e.g., the end of the catheter).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A delivery catheter system comprising:
a catheter assembly including an elongate catheter body having a distal tip portion and a capsule distal to the distal tip portion, said capsule comprising a capsule body capable of containing all or part of a deliverable prosthesis;
a locating member comprising a first portion connected to the capsule body, and an annular second portion extending from the first portion and configured for contacting a valve annulus; and
wherein the annular second portion is moveable from a collapsed state, in which the second annular portion is at least partially retained within a sheath, to a deployed state in which the second annular portion extends radially outwardly from the capsule body.

2. The delivery catheter system according to claim 1, wherein the first portion of the locating member is connected to an external surface of the capsule body, and the locating member is positioned on the capsule body such that a distal edge of the annular second portion is spaced apart from a proximal end of the capsule body by a first distance.

3. The delivery catheter system according to claim 2, wherein the distal edge of the annular second portion is closer to a distal end of the capsule body than the proximal end when the second portion is in the deployed state.

4. The delivery catheter system according to claim 2, wherein the distal edge of the annular second portion in the deployed state is spaced apart from the distal end of the capsule body by a second distance of less than 20 mm.

5. The delivery catheter system according to claim 1, wherein relative movement between the sheath and the capsule in a first direction enables the annular second portion of the locating member to move from the collapsed state to the deployed state.

6. The delivery catheter system according to claim 4, wherein a proximal retraction of the sheath relative to the capsule and/or a distal movement of the capsule relative to the sheath enables the locating member to move from the collapsed state to the deployed state.

7. The delivery catheter system according to claim 1, comprising a deployment arrangement to deploy the prosthesis from the capsule body, said deployment arrangement comprising an actuating arrangement configured to move the prosthesis in a distal direction and a retraction arrangement configured to proximally retract the capsule body.

8. The delivery catheter system according to claim 7, wherein movement of the prosthesis in the distal direction is restricted to a distance less than an elongate length of the capsule body.

9. The delivery catheter system according to claim 7, wherein the locating member is positioned on the capsule body such that a distal edge of the second annular portion is spaced apart from a proximal end of the capsule body by a first distance, and wherein movement of the prosthesis in the distal direction is restricted to a distance substantially equal to the first distance.

10. The delivery catheter system according to claim 7, wherein the distal edge of the second annular portion is spaced apart from the distal end of the capsule body by a second distance, and wherein retraction of the capsule body by a distance at least equal to the second distance enables deployment of the prosthesis.

11. The delivery catheter system according to claim 1, wherein the annular second portion comprises a flexible portion including a flexible brim or skirt.

12. The delivery catheter system according to claim 1, wherein the prosthesis is moveable from a compressed state within the capsule body to an expanded state upon deployment of the prosthesis from the capsule body.

13. The delivery catheter system according to claim 1, wherein the second annular portion is substantially frustoconical.

14. The delivery catheter system according to claim 1, wherein the prosthesis comprises one or more anchor members connecting the prosthesis to the catheter, and wherein the anchor members are positioned on a proximal side of the prosthesis.

15. The delivery catheter system according to claim 1, wherein the locating member comprises a support frame and a porous polymeric membrane covering the support frame.

16. A method of delivering a prosthesis contained within a capsule body of a capsule of a delivery catheter system to a target location within patient's vasculature, the method comprising the steps of:
delivering a catheter system through a patient's vasculature to a desired location with a locating member connected to an external surface of the capsule body in a collapsed state in which the locating member is at least partially retained within a sheath;
at least partially deploying the locating member by a proximal retraction of the sheath;
aligning the locating member with a valve annulus; and
deploying the prosthesis from the capsule body and into position within the valve annulus.

17. The method of claim 16, wherein the step of deploying the prosthesis comprises moving the prosthesis in a distal direction relative to the capsule body.

18. The method of claim 16, wherein the step of deploying the prosthesis comprises proximally retracting the capsule body relative to the prosthesis.
